(19) United States
(12) Patent Application Publication
(10) Pub. No.: US 2012/0093880 A1
(43) Pub. Date: Apr. 19, 2012

(54) METHOD AND KIT FOR TOPICAL ELIMINATION OF PSORIATIC LESIONS WITH A CAUSTIC APPLICATOR

(76) Inventor: Ernest A. Graypel, St. Louis, MO (US)

(21) Appl. No.: 13/251,866

(22) Filed: Oct. 3, 2011

Related U.S. Application Data
(60) Provisional application No. 61/392,664, filed on Oct. 13, 2010.

(51) Int. Cl. A61K 9/00; A61P 17/06; A61K 33/38

(52) U.S. Cl. 424/400; 424/618

(57) ABSTRACT

A method and kit for eliminating a psoriatic lesion involves removal of loose psoriatic scales if present, and moistening remaining scales to a depth to or just above epidermal tissue underlying the scales. The hydrated scales are then contacted with a dry composition of from about 50% to about 95% silver nitrate using a specially configured applicator such that a portion of the moisture of the hydrated psoriatic scales will be absorbed into the dry composition so as to dissolve an outer layer of the silver nitrate into a solution. The solution will then be absorbed into the psoriatic scales to the depth of the moistening thereof to chemically cauterize the moistened scales. The method can be repeated until elimination is achieved. Cauterization of surrounding and underlying tissue is undesirable, and if contacted by moisture, dehydration can be allowed, and the lesion rehydrated as necessary.

This application claims the benefit of U.S. Provisional Application No. 61/392,664, filed Oct. 13, 2010.

### TECHNICAL FIELD

This invention relates generally to treatment of psoriasis, and more particularly to a method and kit for topical elimination of a psoriatic lesion using a caustic applicator, containing in particular, silver nitrate.

### BACKGROUND ART

The disclosure of U.S. Provisional Application No. 61/392,664, filed Oct. 13, 2010, is hereby incorporated herein in its entirety by reference.

Psoriatic lesions of the skin typically comprise reddish, slightly elevated patches or bumps covered with silvery-white scales in the epidermis, referred to as psoriatic plaques. Spots may coalesce into large patches. It is believed that skin injury, infection, stress, and certain drugs may trigger psoriasis. It is reported that in psoriatic lesions, skin cells move at an accelerated rate from the dermis into the epidermis, where they slough off, causing inflammation.

Known treatments for psoriasis typically depend on such factors as patient age, general health and lifestyle, and the severity and location of symptoms influence the type of treatment used to reduce inflammation and decrease the rate at which new skin cells are produced. The course of psoriasis varies with the individual patient. Experimentation is often required, and different treatments are often combined to achieve the best therapeutic response. Generally, known treatments are a function of the severity of the psoriasis. The following is a summary of known psoriasis treatments found in medical literature.

For milder cases, steroid creams and ointments are commonly used. Tazarotene (Tazorac) is found to be effective for mild-to-moderate psoriatic plaques.

Moisturizers and bath oils are used to loosen scales, soften skin, and eliminate the itch. Oatmeal baths can also be helpful. Salicylic acid is also a known treatment to remove dead skin or increase the effectiveness of other therapies.

For more moderate psoriasis, ultraviolet light B (UVB) is used to control psoriasis that covers many areas of the body or that has not responded to topical preparations. Doctors combine UVB treatments with topical medications to treat some patients and sometimes prescribe home phototherapy, in which the patient or caregiver administers the UVB treatments.

For severe psoriasis, Methotrexate (MTX), given as a pill or as an injection can be used. Enbrel can also be prescribed. Still other known medications are efalizumab (Raptiva) and adalimumab (Humira), which suppress the immune system.

Photochemotherapy (PUVA) is a medically supervised procedure that combines medication with exposure to ultraviolet light (UVA) to treat localized or widespread psoriasis.

Non-traditional psoriasis treatments include: soaking in warm water and German chamomile (Matricaria recutita) or bathing in warm salt water; drinking as many as three cups a day of hot tea made with one or a combination of the following herbs: burdock (Arctium lappa) root, dandelion (Taraxacum mongolicum) root, Oregon grape (Mahonia aquifolium), sarsaparilla (Smilax officinalis), and balsam pear (Momardica charantia); taking capsules of evening primrose (Oenothera biennis) oil a day; eating a diet that includes plenty of fish, turkey, celery (for cleansing the kidneys), parsley, lettuce, lemons (for cleansing the liver), limes, fiber, and fruit and vegetable juices; eating a diet that eliminates animal products high in saturated fats, since they promote inflammation; drinking plenty of water; taking nutritional supplements including folic acid, lecithin, vitamin A, vitamin E, selenium, and zinc.

Other reported alternative approaches include identifying and eliminating food allergens from the diet, enhancing the function of the liver, augmenting the hydrochloric acid in the stomach, and completing a detoxification program.

Various shortcomings of the above referenced treatment approaches have been evident, such that for many psoriasis sufferers, satisfactory treatment has not been obtained. This has led the present inventor to investigate possible alternative courses of elimination of psoriatic lesions, particularly topical treatment using chemical cautery agents.

Chemical cautery is a process known for removal of small skin lesions, principally, warts, verrucas, and necrotized tissue, but the literature has not been found to discuss its use in the treatment of psoriasis. In investigating this potential course of treatment, silver nitrate, of the keratolytics medicines group, is of particular interest. Silver nitrate is generally known as an astringent and as having styptic and bactericide properties. In contact with the skin, silver nitrate is known to shrink highly vascular hypergranulation which can be advantageous for delineating skin tissue to be treated from adjacent healthy tissue, such as nail borders and sulcus in onychocryptosis. Silver nitrate is also known for use to treat pyogenic granuloma. For hemostasis, silver nitrate is used to stem hemorrhage from exposed blood vessels in the nasal passages, and exposed blood vessels in the skin after injury or trauma, e.g., abrasions, etc., resection or removal of growths and the like. In this latter regard, the literature teaches that the silver nitrate is to be applied only to the bleeding blood vessels, not the lesion overall or generally.

Silver nitrate is soluble in water, and for topical application, is found commercially available in pencil and stick applicator form. In both instances, the tip of the device, e.g., pencil or applicator, contains or is coated with the silver nitrate is relatively small, to facilitate limiting contact to just the bleeding blood vessel and closely surrounding tissue, and the literature warns that contact with surrounding healthy tissue is to be avoided as injury can occur. Caustic pencils and stick applicators having a concentration of about 50 percent to about 95 percent silver nitrate are available commercially. Reference in this regard, a commercially available caustic pencil comprising a compound of 200 mg silver nitrate and 300 mg potassium nitrate, available under the trade name Surgipack, from Tyco Healthcare Group of the United Kingdom. Directions for use contained on the packaging of that product are as follows.

1. Apply with caution as a Caustic Pencil will burn skin and stain most surfaces.

2. Protect skin around wart with petroleum jelly. Moistened tip of pencil and water-do not lick!Wipe excess liquid using a tissue. Discard used tissue immediately.

3. Apply moistened tip to wart GENTLY to ensure the tip does not break as CAUSTIC PENCILS ARE BRITTLE. Apply for 1-2 minutes only. Repeat if necessary after 24 hours. Do not use more than 3 times.

4. Cover with a dry dressing.

5. Using a tissue, dry tip before replacing the cap.

A cautionary note on the product packaging indicates that it should not be used on moles, birthmarks or unusual skin growths. The Surgipack product makes no mention or suggestion for use as a treatment for psoriasis.

Other literature instructs that the tip of the pencil or applicator is applied to dampened lesions in small circular motions or twirled to dissolve the chemical and work up to the maximum concentration.

It is also known to use a 25 percent silver nitrate solution to treat dorsal nucleated lesions also referred to as neurovascular corns. However, again there is no mention or suggestion for treatment of psoriasis.

### SUMMARY OF THE INVENTION

What is disclosed is a method and kit for topical elimination of a psoriatic lesion using a caustic applicator, containing in particular, silver nitrate preferably in the commercially available quantities, that is, in a concentration of between about 50 and about 95 percent in solid form. Preferably also, the applicator has a surface area containing the silver nitrate having a size of at least about 1 cm², to enable application of the silver nitrate in a particular controlled manner, to destroy the affected epidermal cells while minimizing damage to and preserving surrounding tissue, particularly the tissue underlying the affected tissue. The unexpected discovery of the efficacy of the invention is based upon a conclusion drawn from several observations, as follows.

It has been observed that those conditions successfully treated with chemical cauterization, namely, warts, verrucas, and other similar conditions, are essentially parasitic conditions, namely, a viral source ensconced in the surface of the epidermis and which beneficially thrives on nutrients present in that host environment. With these conditions, the cells of the region of the epidermis on which such conditions exist are relatively healthy. Psoriasis, on the other hand, is a condition of, or within, the outer skin cells of the epidermis itself, wherein those cells die and result in the familiar scales and irritating itchiness. It is presently unknown if psoriasis is a result of a defect of the cells of the epidermis itself, an autoimmune condition or bacterial condition, or is the result of a combination of conditions. Contrast hypergranulation which is a hyper growth condition of the cells that can result from various sources.

It has been further observed that psoriasis presents in only about the outer one third or so cells of the epidermis, yet can be quite widespread, that is, the plaques or lesions can cover a relatively large skin area. It has also been observed that the underlying approximately two thirds of the epidermis and dermis appear healthy and unaffected by the condition. It is thus surmised that the condition is limited to the outermost one third portion of the epidermis. Therefore, a premise of the invention is to eradicate the condition by controlled chemical cauterization of the affected cells of the approximate outer third of the epidermis, without invoking a scarring response or damaging the underlying epidermal tissue.

Electrical cauterization has been studied as a possible approach, but deemed less desirable due to a difficulty in limiting heat generated during the process to just the psoriatic tissue, and the potential for triggering a scar tissue response. It has been found that chemical cauterization, on the other hand, can be more precisely limited to the affected tissue. That is, if applied in a controlled manner, penetration of the cauterizing chemical to the underlying healthy tissue of the epidermis can be prevented. Unlike the thermal transfer aspect of electrical cauterization, the chemical cauterization effects can be contained or limited, particularly, by the limiting the extent of hydration of the affected epidermal tissue, which activates and carries the cauterizing agent (silver nitrate) into the tissue to be treated.

To discover a suitable delivery mechanism for the silver nitrate which satisfactorily limits penetration of the chemical cauterizing agent into the underlying tissue, experiments were conducted using several silver nitrate compositions. In one experiment, a suspension of less than 50 percent silver nitrate (by concentration) in oil was prepared and applied directly to psoriatic plaques using a syringe. This yielded less than satisfactory elimination of the psoriasis, with some lesions remaining, which was deemed the result of uneven coverage obtained by this delivery mechanism.

In another experiment, a 95 percent silver nitrate pencil having an effective applicator surface area of between about 4 and 5 mm was used to apply the silver nitrate to lightly moistened psoriatic plaques. This yielded better results, with a higher percentage of elimination. But, the small applicator area of the pencil made achieving adequate, even coverage of larger plaques time consuming and difficult, such that remoistening areas of the larger psoriatic plaque was required to avoid uneven application and spotty psoriasis elimination.

It was therefore determined from observed results of the above experiments, that a larger applicator, on the order of about 1 cm² or larger, would be the preferred mechanism of applying the silver nitrate, in a dry applicator concentration of from about 50 percent to about 95 percent. It was also determined that an applicator surface containing the silver nitrate having a convex shape would be advantageous to enable applying the silver nitrate in a slight rolling action, to enable manipulating the applicator for following irregular edges of the plaques, to apply the silver nitrate evenly, and to avoid applying it to adjacent healthy skin. The silver nitrate can be affixed to the surface of the applicator in a suitable commercially known manner, such as that used for affixing it to known applicator sticks, and it will be supported by the applicator so as to be less susceptible to breakage and crumbling. It is contemplated according to the invention to supply the silver nitrate applicator in kit form, including also instructions for preparing the lesion or lesions for treatment and performing the treatment.

To effect treatment, the affected psoriatic plaques or lesions are first moistened for a few moments, to an extent such that excessive scales are removed and the remaining scales appear hydrated. There should be no moistening of adjacent healthy skin tissue. The applicator, in a dry state, is then positioned in contact with the plaque or lesion to be treated, and is rolled in an appropriate manner and direction or directions, to make momentary contact with the entire surface or the plaque or lesion in a manner that a portion of the moisture contained in the scales is transferred to the silver nitrate on the surface of the applicator, sufficient for dissolving a thin layer of the silver nitrate and transferring just the layer of dissolved silver nitrate to the surface of the plaque or lesion. The moisture contained in the psoriatic tissue will then transfer the silver nitrate through the scales to the lowermost affected tissue, via capillarity, as the chemical cauterization reaction of the silver nitrate occurs. The treated lesions can be left exposed to air, and will dehydrate over time, so as to shed the scales and dead psoriatic tissue, leaving the healthy underlying tissue to heal and replace the psoriatic tissue with healthy skin cells by the normal processes of tissue regeneration. Typically, psoriatic tissue is eliminated with a single treatment, with no scarring. If necessary, a second treatment can be performed.

As a supplemental step, after hydration of the affected tissue to the desired depth, it may be advantageous to momentarily remoisten the surface of the lesion or plaque, to provide additional available moisture for dissolving the solid silver nitrate. Again, it is desired to avoid chemical cauterization of adjacent healthy skin tissue, and by hydrating only the diseased tissue and not the applicator itself, in the event the applicator contacts healthy skin, because the silver nitrate is not activated, no harm will occur.

### BRIEF DESCRIPTION OF THE DRAWINGS

FIG. 1 is a perspective view of a human hand afflicted with psoriatic lesions;

FIG. 2 is a side view of a human arm afflicted with representative larger psoriatic lesions, and showing a manner of moistening the lesions;

FIG. 3 is a side view of a human arm afflicted with representative large psoriatic lesions, particularly about the elbow;

FIG. 4 is a side view of an applicator for topical application of silver nitrate for elimination of psoriatic lesions according to the invention;

FIG. 5 is a perspective view of the applicator of FIG. 4;

FIG. 6 illustrates use of the applicator for applying silver nitrate to psoriatic lesions according to the invention;

FIG. 7 illustrates a further step of use of the applicator according to the invention;

FIG. 8 illustrates a further step of use of the applicator according to the invention; and

FIG. 9 is a list of steps of an embodiment of the method of the invention for inclusion with the applicator of FIG. 4 in a kit for the elimination of psoriatic lesions.

### DETAILED DESCRIPTION OF THE INVENTION

Referring now to the figures, a preferred method and kit of the invention for eliminating psoriatic plaques or lesions will be described. In FIG.

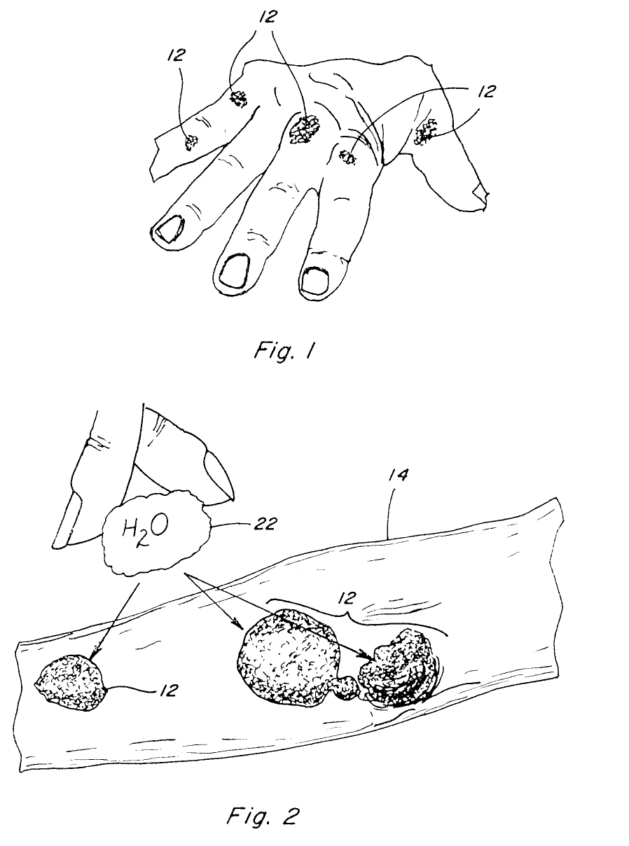

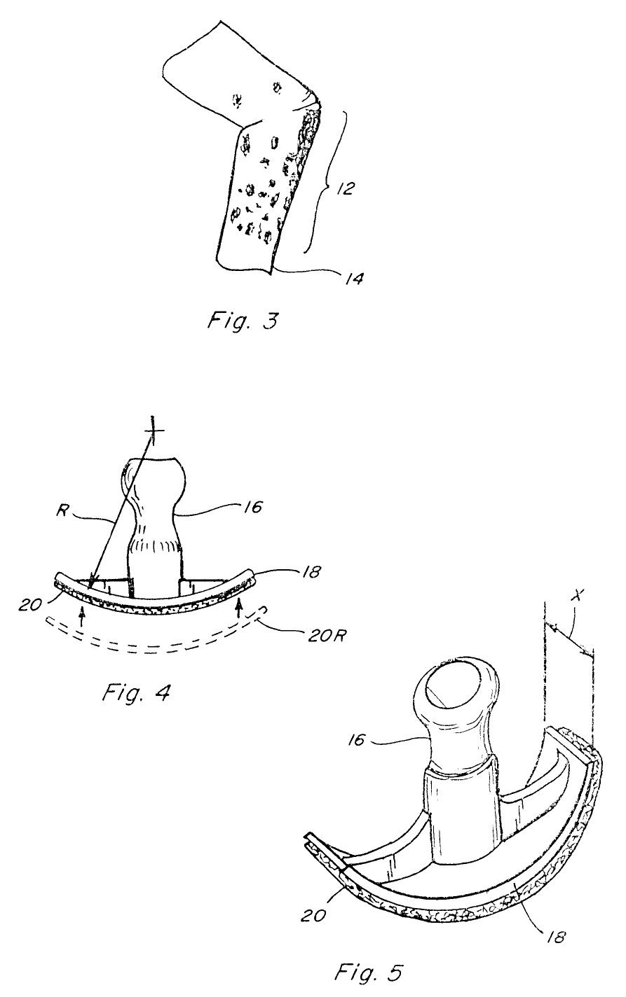

1 a human hand 10 including representative psoriatic lesions 12 is shown. In FIGS. 2 and 3, a human arm 14 afflicted with representative larger psoriatic lesions 12, of an extent that can vary from less than a centimeter to several centimeters, is shown.

FIGS. 4 and 5 show a side view and a perspective view of a representative applicator 16 for topical application of silver nitrate for elimination of psoriatic lesions according to the invention. Applicator has a convex curved pad 18 of rigid or semi-rigid construction, having an outer surface including a thin layer 20 comprising water soluble silver nitrate affixed thereto. The concentration of silver nitrate of layer 20 is preferably from about 50 percent to about 95 percent, in mixture with a suitable binder such as potassium nitrate. Layer 20 can be suitably mounted to the surface of pad 18 in any suitable, conventional manner, such as with a layer of adhesive, mechanical restraining mechanism, such as clips, or pins, fusion, or the like, and can be replaceable, as illustrated by layer 20R in FIG. 4. The surface of layer 20 will have an extent in at least one direction, denoted by distance X in FIG. 5, of at least about 1 cm, with a minimum preferred overall size of at least about 1 cm².

Layer 20 of silver nitrate is shown in the exemplary construction as having a rounded overall shape. It should be noted, however, that it can have a variety of shapes, such as oval, hourglass, irregular, or polygonal, such as, but not limited to, square, rectangular, diamond, or the like, or a combination of any of these shapes, as desired or required for a particular application. As another feature of the invention, layer 20 of silver nitrate can have a convex shape, preferably with a radius of curvature R of several centimeters, as illustrated in FIG. 4. Applicator 16 can include a handle having any of a variety of configurations, here being a knob suitable for holding between the fingers.

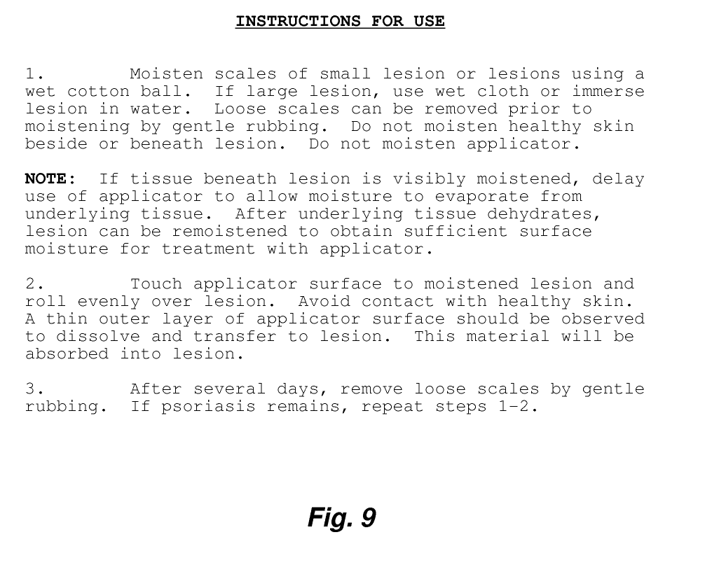

FIGS. 2, 6, 7, 8 and 9 illustrate steps of treatment of a representative variety of lesions 12 using applicator 16. As a first step as illustrated in FIGS. 2 and 9, lesions 12 are moistened in a suitable manner, for instance, but not limited to using a moistened cotton ball 22. Alternatively, the affected limb or body portion can be immersed in water. The duration of moistening will be sufficient to effect absorption of moisture throughout the psoriatic tissue, but not substantially deeper, which, as explained above, will typically encompass about the outer one third of the epidermis. The moistening will be determined visually, by observing the outward appearance of the moistened tissue being evenly covered by silver nitrate. If it is observed that moisture absorption has occurred too deeply, application of the silver nitrate can be delayed to allow evaporation or wicking of moisture from the underlying epidermal tissue sufficiently. It may, however, be advantageous to remoisten the surface of the lesion after dehydrating the underlying tissue, to provide sufficient surface moisture for creating a desired amount of dissolved silver nitrate for penetration into the lesion.

Once the lesions or plaques to be treated have been moistened to the desired extent, applicator 16 is used to apply the silver nitrate by gently bringing the surface of layer 20 into contact with the moistened psoriatic tissue in a manner to cause a portion of the outer layer of the silver nitrate to be dissolved sufficiently such that a small quantity of the dissolved silver nitrate solution will be transferred to the psoriatic tissue, and by capillarity, throughout that tissue sufficiently for thorough chemical cauterization thereof.

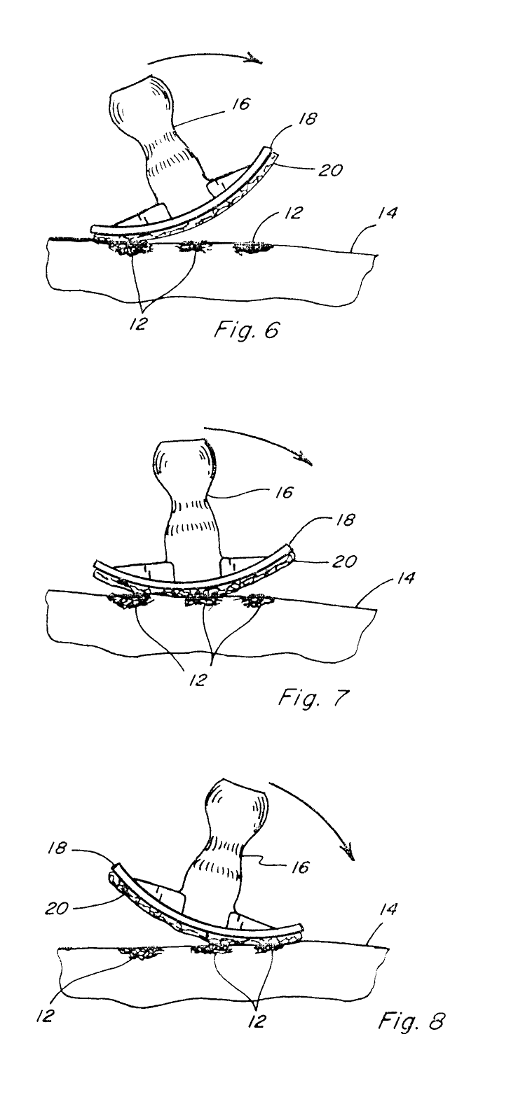

FIGS. 6, 7 and 8 illustrate, and FIG. 9 explains how applicator 16 can be manipulated in a rolling action to contact the surface of silver nitrate layer 20 with the affected tissue. In this manner, contact between the silver nitrate and adjacent healthy tissue can be avoided. Also, in the event of contact between healthy skin and the silver nitrate, if the healthy skin is not moistened, injury will not occur, as the silver nitrate on that portion of the applicator will not be activated. These FIGURES also illustrate that, by virtue of the larger surface area of applicator 16, a corresponding larger area of a lesion can be treated simultaneously and uniformly, compared to what is possible using a smaller applicator as known in the prior art, without injury to adjacent healthy skin.

As an advantage of the invention, applicator 16 can be provided in kit form including the instructions of FIG. 9 or similar instructions, and optional wetting means such as a cotton ball or balls 22, and optionally gauze for covering the treated site.

In light of all the foregoing, it should thus be apparent to those skilled in the art that there has been shown and described a novel method of topical elimination of psoriatic lesions with caustic applicator. However, it should also be apparent that, within the principles and scope of the invention, many changes are possible and contemplated, including in the details, materials, and arrangements of parts which have been described and illustrated to explain the nature of the invention. Thus, while the foregoing description and discussion addresses certain preferred embodiments or elements of the invention, it should further be understood that concepts of the invention, as based upon the foregoing description and discussion, may be readily incorporated into or employed in other embodiments and constructions without departing from the scope of the invention.

What is claimed is:

1. A method for eliminating a psoriatic lesion from a skin surface, comprising steps of:
moistening the lesion to hydrate psoriatic scales thereof, while avoiding moistening adjacent regions of the skin surface;
contacting the hydrated psoriatic scales with a dry composition of from about 50% to about 95% silver nitrate to transfer a portion of the moisture of the hydrated psoriatic scales to the dry composition in a manner to dissolve and release a layer of the silver nitrate from the dry composition and transfer the dissolved silver nitrate to the hydrated psoriatic scales of the lesion such that the silver nitrate will be absorbed thereby and will chemically cauterize the hydrated scales.

2. The method of claim 1, wherein the step of hydrating the psoriatic scales is ceased prior to hydrating tissue underlying the psoriatic scales.

3. The method of claim 1, wherein after the step of hydrating the psoriatic scales, if tissue underlying the psoriatic scales has been hydrated also, then delaying the step of contacting the hydrated psoriatic scales with the dry composition sufficiently to allow the tissue underlying the psoriatic scales to dehydrate.

4. The method of claim 1, wherein the dry composition comprises from about 50% to about 95% silver nitrate and has a surface area of at least about 1 cm².

5. The method of claim 4, wherein the dry composition has a convex shape.

6. The method of claim 5, wherein the step of contacting the hydrated psoriatic scales with the dry composition of the silver nitrate comprises rolling or rocking the dry composition in contact with the hydrated scales.

7. The method of claim 1, wherein the step of dissolving and releasing the layer of the silver nitrate from the dry composition and transferring the dissolved silver nitrate to the hydrated psoriatic scales forms a solution of the silver nitrate and the moisture and absorbing the solution into the hydrated scales to chemically cauterize the hydrated scales.

8. The method of claim 1, wherein the steps of moistening the lesion and contacting the hydrated psoriatic scales with the dry composition, are repeated after a period sufficient to allow the cauterized scales to dehydrate and detach from the underlying tissue, until the lesion is eliminated.

9. The method of claim 1, comprising a step prior to the step of moistening the psoriatic scales of the lesion, of removing any loose ones of the psoriatic scales from the lesion.

10. A method of chemical cauterizing a psoriatic lesion on a skin surface of a living body, comprising steps of:
removing loose psoriatic scales from the lesion and moistening remaining psoriatic scales of the lesion to a depth to or just above epidermal tissue underlying the scales;
contacting the hydrated psoriatic scales with a dry composition of from about 50% to about 95% silver nitrate in a manner such that a portion of the moisture of the hydrated psoriatic scales will be absorbed into the dry composition so as to dissolve an outer layer of the silver nitrate into a solution that will then be absorbed into the psoriatic scales to the depth of the moistening thereof to chemically cauterize the moistened scales.

11. The method of claim 10, wherein after the step of moistening remaining psoriatic scales of the lesion, if the epidermal tissue underlying the psoriatic scales has been hydrated, then delaying the step of contacting the hydrated psoriatic scales with the dry composition, to allow the tissue to dehydrate.

12. The method of claim 10, wherein the dry composition comprises from about 50% to about 95% silver nitrate and has a surface area of at least about 1 cm².

13. The method of claim 12, wherein the dry composition has a convex shape.

14. The method of claim 13, wherein the step of contacting the hydrated psoriatic scales with the dry composition of the silver nitrate comprises rolling or rocking the dry composition in contact with the hydrated scales such that the solution will be absorbed into the psoriatic scales substantially evenly over an outer surface thereof.

15. The method of claim 10, wherein the steps of moistening the psoriatic scales of the lesion and contacting the hydrated psoriatic scales with the dry composition, are repeated after a period sufficient to allow the cauterized scales to dry and be removed from the underlying tissue, until the lesion is eliminated.

16. The method of claim 10, comprising a step after the step of moistening the psoriatic scales of the lesion and before the step of contacting the hydrated psoriatic scales with the dry composition, of moistening only an outer portion of the psoriatic scales of the lesion, to provide additional moisture thereon for absorption into the dry composition.

17. A method of chemical cauterizing a psoriatic lesion on a skin surface of a living body, comprising steps of:
providing an applicator having a concave outer surface having a surface area of at least about 1 cm² covered with a dry composition comprising from about 50% to about 95% silver nitrate;
moistening psoriatic scales of the lesion to a depth to or just above epidermal tissue underlying the scales;
contacting the hydrated psoriatic scales with the dry composition of from about 50% to about 95% silver nitrate in a rolling manner such that a portion of the moisture of the hydrated psoriatic scales will be absorbed into the dry composition so as to dissolve an outer layer of the silver nitrate into a solution that will then be absorbed into the psoriatic scales to the depth of the moistening thereof to chemically cauterize the moistened scales.

18. The method of claim 17, wherein after the step of moistening the psoriatic scales of the lesion, if the epidermal tissue underlying the psoriatic scales has been hydrated, then delaying the step of contacting the hydrated psoriatic scales with the dry composition, to allow the tissue to dehydrate.

19. The method of claim 10, wherein the steps of moistening the psoriatic scales of the lesion and contacting the hydrated psoriatic scales with the dry composition, are repeated after a period sufficient to allow the cauterized scales to dry and be removed from the underlying tissue, until the lesion is eliminated.

20. The method of claim 17, comprising a step after the step of moistening the psoriatic scales of the lesion and before the step of contacting the hydrated psoriatic scales with the dry composition, of moistening only an outer portion of the psoriatic scales of the lesion, to provide additional moisture thereon for absorption into the dry composition.

21. A kit for chemical cauterizing a psoriatic lesion on a skin surface of a living body, comprising:
an applicator having a convex outer surface having a surface area of at least about 1 cm² covered with a dry composition comprising from about 50% to about 95% silver nitrate; and
written instructions for using the applicator comprising steps including:
moisten scales of small lesion or lesions using a wet cotton ball; loose scales can be removed prior to moistening by gentle rubbing; do not moisten healthy skin beside or beneath lesion; do not directly moisten applicator;
if tissue beneath lesion is visibly moistened, delay use of applicator to allow moisture to evaporate from underlying tissue; after underlying tissue dehydrates, lesion can be remoistened to obtain sufficient surface moisture for treatment with applicator; and
touch applicator surface to moistened lesion and roll evenly over lesion; avoid contact with healthy skin; a thin outer layer of applicator surface should be observed to dissolve and transfer to lesion; allow to dry.
